(19) United States
(12) Patent Application Publication
(10) Pub. No.: US 2018/0348218 A1
(43) Pub. Date: Dec. 6, 2018

(54) SEROLOGY ASSAY FOR RECENT MALARIA INFECTION

(71) Applicants: Tokitae LLC, Bellevue, WA (US); London School of Hygiene & Tropical Medicine, London (GB)

(72) Inventors: David Michael Cate, Bellevue, WA (US); Christopher John Drakeley, Ashford (GB); Bryan Ross Greenhouse, San Francisco, CA (US); Kevin Paul Flood Nichols, Issaquah, WA (US); Isabel Rodriguez-Barraquer, San Francisco, CA (US); Kevin Kweku Adjei Tetteh, London (GB); Bernhard Hans Weigl, Seattle, WA (US)

(21) Appl. No.: 15/991,355

(22) Filed: May 29, 2018

Related U.S. Application Data
(60) Provisional application No. 62/515,233, filed on Jun. 5, 2017.

(51) Int. Cl. G01N 33/569; G01N 33/564; G01N 33/577; G01N 33/543

(52) U.S. Cl. CPC G01N 33/56905; G01N 33/564; G01N 2800/26; G01N 33/543; G01N 2333/445; G01N 33/577

(57) ABSTRACT

In some embodiments, an immunoassay device for detection of recent malaria infection includes: a sample pad positioned adjacent to a first end of a conjugate pad, wherein either the sample pad or the first end of the conjugate pad include a detection complex able to bind anti-ETRAMP5 antibodies; wherein a second end of the conjugate pad includes one or more areas impregnated with a construct protein including ETRAMP5 protein fragments. In some embodiments, the detection complex for anti-ETRAMP5 antibodies includes a capture particle complexed with anti-IgG antibodies. In some embodiments, the detection complex for anti-ETRAMP5 antibodies includes a detection particle complexed with a complex molecule including ETRAMP5 protein fragments.

If an Application Data Sheet (ADS) has been filed on the filing date of this application, it is incorporated by reference herein. Any applications claimed on the ADS for priority under 35 U.S.C. §§ 119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC § 119(e) for provisional patent applications, for any and all parent, grandparent, great-grandparent, etc. applications of the Priority Application(s)).

### PRIORITY APPLICATIONS

The present application claims benefit of priority of U.S. Provisional Patent Application No. 62/515,233, entitled SEROLOGY ASSAY FOR RECENT MALARIA INFECTION, naming DAVID MICHAEL CATE, CHRISTOPHER JOHN DRAKELEY, BRYAN ROSS GREENHOUSE, KEVIN PAUL FLOOD NICHOLS, ISABEL RODRIGUEZ-BARRAQUER, KEVIN KWEKU ADJEI TETTEH, AND BERNHARD HANS WEIGL as inventors, filed 5 Jun. 2017, which was filed within the twelve months preceding the filing date of the present application or is an application of which a currently co-pending priority application is entitled to the benefit of the filing date.

If the listings of applications provided above are inconsistent with the listings provided via an ADS, it is the intent of the Applicant to claim priority to each application that appears in the Domestic Benefit/National Stage Information section of the ADS and to each application that appears in the Priority Applications section of this application.

All subject matter of the Priority Applications and of any and all applications related to the Priority Applications by priority claims (directly or indirectly), including any priority claims made and subject matter incorporated by reference therein as of the filing date of the instant application, is incorporated herein by reference to the extent such subject matter is not inconsistent herewith.

### SUMMARY

In some embodiments, an immunoassay device for detection of recent malaria infection includes: a sample pad positioned adjacent to a first end of a conjugate pad, wherein either the sample pad or the first end of the conjugate pad include a detection complex able to bind anti-ETRAMP5 antibodies; wherein a second end of the conjugate pad includes one or more areas impregnated with a construct protein including ETRAMP5 protein fragments. In some embodiments, the detection complex for anti-ETRAMP5 antibodies includes a capture particle complexed with anti-IgG antibodies. In some embodiments, the detection complex for anti-ETRAMP5 antibodies includes a detection particle complexed with a complex molecule including ETRAMP5 protein fragments.

In some embodiments, an immunoassay device for detection of recent malaria infection includes: a sample pad positioned adjacent to a first end of a conjugate pad, wherein either the sample pad or the first end of the conjugate pad include a first detection complex able to bind human anti-ETRAMP5 antibodies and a second detection complex able to bind to a malaria immune response antigen; wherein a second end of the conjugate pad includes a first region including a first capture particle complexed with a complex molecule including ETRAMP5 protein fragments, and a second region including a second capture particle complexed with a complex molecule including one or more fragments of the malaria immune response protein.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a schematic of an immunoassay.

FIG. 2 is a schematic of another immunoassay.

FIG. 3 is a schematic of an immunoassay in a lateral flow embodiment in a top-down view.

FIG. 4 is a schematic of another immunoassay in a lateral flow embodiment in a traverse side view.

FIG. 5 is a schematic of an immunoassay.

FIG. 6 is a schematic of a set of protein constructs.

FIG. 7 is a schematic of a set of protein constructs.

FIG. 8 illustrates malaria antigens test results from population cohorts.

FIG. 9 illustrates malaria antigens test results from population cohorts.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Detection of malaria by a serology assay, such as an immunoassay, in malaria-endemic regions of the world is made more complex by the ongoing presence of antibodies to malaria proteins in individuals who have suffered from malaria at some time in the past. People living in malaria-prevalent regions often are infected multiple times in their lives, possibly with multiple distinct infection events in the same season or year. Many biological markers of malaria have the limitation that they will continue to provide positive results in a serology assay for some time after the active infection has ended due to lingering immune response in infected individuals. Depending on the biological marker of malaria infection, assay results can continue to be positive for months to years. In regions where malaria is endemic, many individuals have been infected multiple times during their lifetimes and can be infected multiple times in the same year or season. Serology assays on body fluids, such as blood, taken from such individuals can show persistent positive results in some assays for months to years after an active malaria infection has ended. See Helb et al., Novel Serologic Biomarkers Provide Accurate Estimates of Recent Plasmodium Falciparum Exposure for Individuals and Communities, PNAS doi 10.1073, published online Jul. 27, 2015, which is incorporated by reference.

In order to detect recent infection and to inform medical diagnosis and/or provide data to epidemiology studies of malaria incidence, immunoassays as described herein detect biological markers that are specific to recent malaria infection. More specifically, immunoassays as described herein detect human antibodies to the malarial early transcribed membrane protein 5 (as used herein “ETRAMP5”). ETRAMP5 is expressed by a malaria parasite during early stage infection of Plasmodium vivax, and recently infected humans develop antibodies to this protein. Generally, antibodies to ETRAMP5 protein are only detectable in humans within a several month period after infection. Immunoassays detecting antibodies reacting with portions of the ETRAMP5 protein, therefore, specifically indicate a relatively recent infection.

In some embodiments, it is expected that the immunoassays will only yield positive results on samples from individuals who have been infected with malaria within a period less than one year. In some embodiments, it is expected that the immunoassays will only yield positive results on samples from individuals who have been infected with malaria within a period less than six months. In some embodiments, it is expected that the immunoassays will only yield positive results on samples from individuals who have been infected with malaria within a period less than three months. The period of time since infection for a particular immunoassay embodiment is dependent on factors including the concentration of construct protein at the visualization line of the assay, the volume of sample used in the assay, the fragment(s) of ETRAMP5 protein selected, and buffer conditions of the assay. Some embodiments utilize multiple ETRAP5 protein fragments to improve accuracy of the test. Some embodiments utilize ETRAP5 protein fragments in combination with fragments of other proteins.

Devices for detection of recent malaria infection can include immunoassay devices, such as lateral flow assay (LFA) devices. Devices are generally single use and disposable, in order to minimize the risk of cross-contamination and/or infection of clinical personnel. Serology assays for malaria often use blood as a sample, ideally volumes on the order of a few drops to minimize patient intervention. Blood can be obtained, for example, from a small finger prick and placed directly on the assay. Preferably immunoassays require minimal handling by clinical or lab personnel and deliver results in less than one hour.

In some embodiments, an immunoassay device configured to detect recent malaria parasite infection includes: a sample pad positioned adjacent to a first end of a conjugate pad, wherein either the sample pad or the first end of the conjugate pad include a detection complex able to bind anti-ETRAMP5 antibodies; wherein a second end of the conjugate pad includes one or more areas impregnated with a construct protein including ETRAMP5 protein fragments. During use, a human antibody binds both the detection complex with anti-ETRAMP5 antibodies from a positive sample and the construct protein including ETRAMP5 protein fragments simultaneously, creating a positive signal at the location of the construct protein including ETRAMP5 protein fragments on the conjugate pad. In some embodiments, the detection complex for anti-ETRAMP5 antibodies includes a capture particle complexed with anti-IgG antibodies. In some embodiments, the detection complex for anti-ETRAMP5 antibodies includes a detection particle complexed with a complex molecule including ETRAMP5 protein fragments.

Depending on the embodiment, the construct protein including ETRAMP5 protein fragments can include a protein corresponding to exon 1 of the ETRAMP5 DNA sequence, and/or a protein corresponding to a subunit of exon 1. In some embodiments, the construct protein including ETRAMP5 protein fragments can include a protein corresponding to exon 1 of the ETRAMP5 genomic DNA sequence or a fragment thereof and a protein used as a tag molecule. For example, a construct protein including ETRAMP5 protein fragments can include a protein corresponding to exon 1 of the ETRAMP5 genomic DNA sequence or a fragment thereof and a Glutathione S-transferase (GST) protein from Schistosoma japonicum. For example, a construct protein including ETRAMP5 protein fragments can include a protein corresponding to exon 1 of the ETRAMP5 genomic DNA sequence or a fragment thereof and a human Glutathione S-transferase (GST) protein. For example, a construct protein including ETRAMP5 protein fragments can include a protein corresponding to exon 1 of the ETRAMP5 genomic DNA sequence or a fragment thereof and a chain of at least six histidine molecules, such as six histidine molecules, seven histidine molecules, eight histidine molecules, nine histidine molecules, ten histidine molecules, and so on. For example, a construct protein including ETRAMP5 protein fragments can include a protein corresponding to exon 1 of the ETRAMP5 genomic DNA sequence or a fragment thereof and a protein including a portion of carbohydrate binding molecule 2 (CBM2) protein. In some embodiments, the construct protein including ETRAMP5 protein fragments is purified from E. Coli. In some embodiments, the construct protein including ETRAMP5 protein fragments is purified from mammalian cells.

Some embodiments include a first capture particle complexed with a complex molecule including ETRAMP5 protein fragments, and a second capture particle complexed with a complex molecule including a non-ETRAMP5 malaria immune response antigen. The capture particles can be positioned in different regions of the assay. For example, a conjugate pad can be impregnated with the first capture particle in a first region and impregnated with a second capture particle in a second region distal to the first. In some embodiments, the first capture particle complexed with a complex molecule including ETRAMP5 protein fragments includes a protein construct of one or more ETRAMP protein fragments attached in a series. For example, depending on the embodiment a non-ETRAMP5 malaria immune response antigen can include at least one of: carbohydrate-binding module family 2 (CBM2), gametocyte exported protein (GEXP), merozoite surface protein 1 (MSP1), merozoite surface protein 2 (MSP2), glutamate-rich protein (GLURP), circumsporozoite protein (CSP), and/or reticulocyte-binding protein (Rh5). A non-ETRAMP5 malaria immune response antigen can include a fragment of one or more of these proteins. For example, a MSP2 antigen can include a MSP2.CH150.0, and/or a MSP2.Dd2.KT protein fragment.

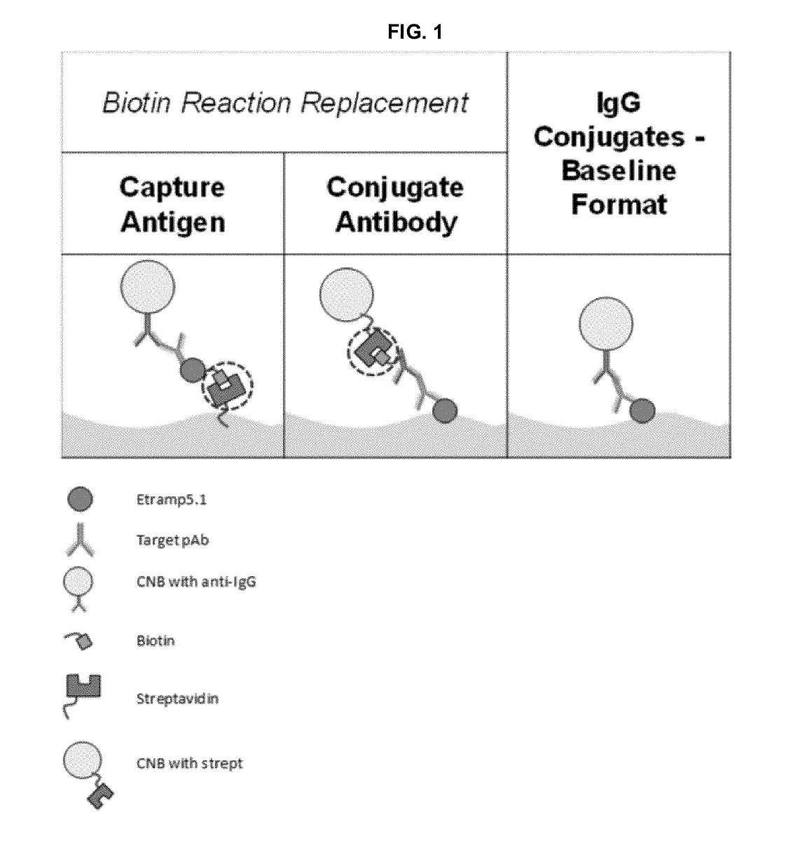

FIG. 1 depicts a schematic of an immunoassay reaction comparing a system dependent on biotin with a system wherein the detection complex for anti-ETRAMP5 antibodies includes a detection particle complexed with anti-IgG antibodies. In the diagram, the large circle represents a detection molecule, such as a gold particle, latex bead, or similar particle used for visualization in immunoassays. The detection molecule can be coated with anti-IgG antibodies, although for the purposes of illustration it is shown as a single Y-shaped anti-IgG antibody. The graphic panel on the right depicts a representative detection particle affixed to a representative IgG antibody. In a device, the detection particle would be coated with multiple anti-IgG antibodies. The IgG antibody affixed to the detection particle is complexed with a target antibody, a human antibody against a malaria parasite early infection protein such as ETRAMP5. The lower portion of the panel depicts the second end of the conjugate pad, shown as a curved surface at the lower portion of the panel, in an area impregnated with a construct protein including ETRAMP5 protein fragments, shown as the smaller circle (“Etramp5.1”). The construct protein includes ETRAMP5 protein fragments from exon 1 of the DNA sequence. The target antibody from the sample forms a complex with both the detection molecule coated with anti-IgG antibodies and the construct protein containing at least one ETRAMP5 segment from exon 1 of the DNA sequence.

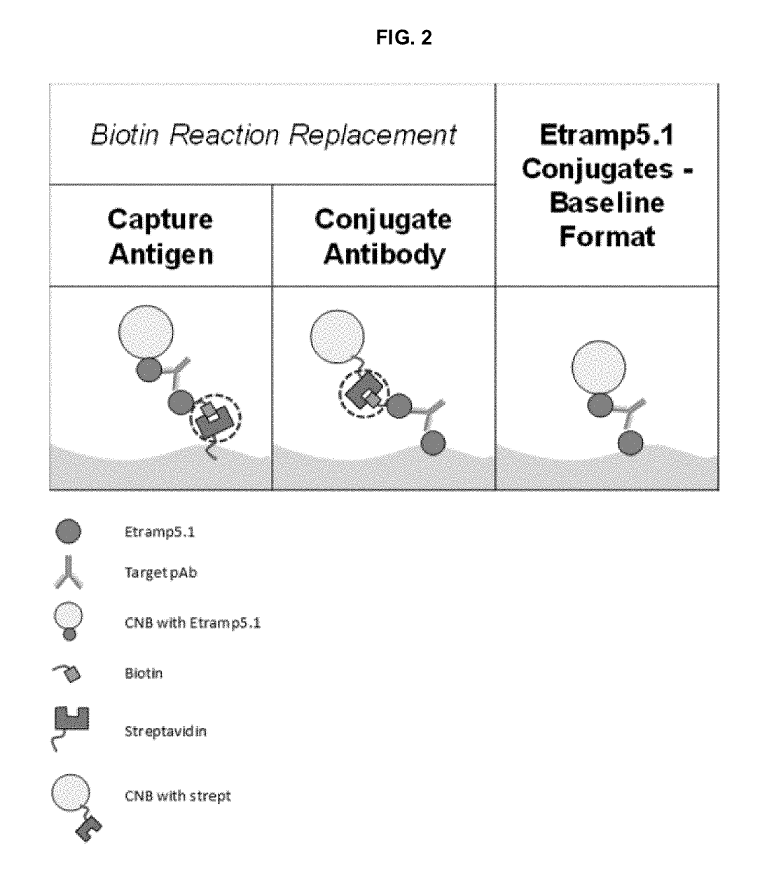

FIG. 2 depicts a comparison of schematic of an immunoassay reaction comparing a system dependent on biotin with a system wherein the detection complex for anti-ETRAMP5 antibodies includes a detection particle complexed with one or more ETRAMP5 protein fragments. In the diagram, the large circle represents a detection molecule, such as a gold particle, latex bead, or similar particle used for visualization in immunoassays. The detection molecule can be coated with ETRAMP5 protein fragments, particularly the protein encoded by exon 1 or fragments thereof. For the purposes of illustration the ETRAMP5 coating is shown as a single small fragment, the smaller dark circle of the illustration (“Etramp5.1”). The graphic panel on the right depicts a representative detection particle affixed to a representative ETRAMP5 protein fragment coating. In a device, the detection particle would be coated with multiple ETRAMP5 protein fragments. The ETRAMP5 protein fragment affixed to the detection particle is complexed with a target antibody, a human antibody against the malaria parasite early infection protein ETRAMP5. The lower portion of the panel depicts the second end of the conjugate pad, shown as a curved surface at the lower portion of the panel, in an area impregnated with a construct protein including ETRAMP5 protein fragments, shown as the smaller dark circle. The construct protein includes ETRAMP5 protein fragments from exon 1 of the DNA sequence. The target antibody from the sample forms a complex with both the detection molecule coated with ETRAMP5 protein fragments and the construct protein containing at least one ETRAMP5 fragment from exon 1 of the DNA sequence. The target antibody from the sample binds to both the detection molecule coated with ETRAMP5 protein fragments and the construct protein containing at least one ETRAMP5 fragment simultaneously, creating a detectable complex at a positive result region when such antibodies are present. In some embodiments, a positive result is visual to the eye of an observer, while in others it is detectable using a reader device.

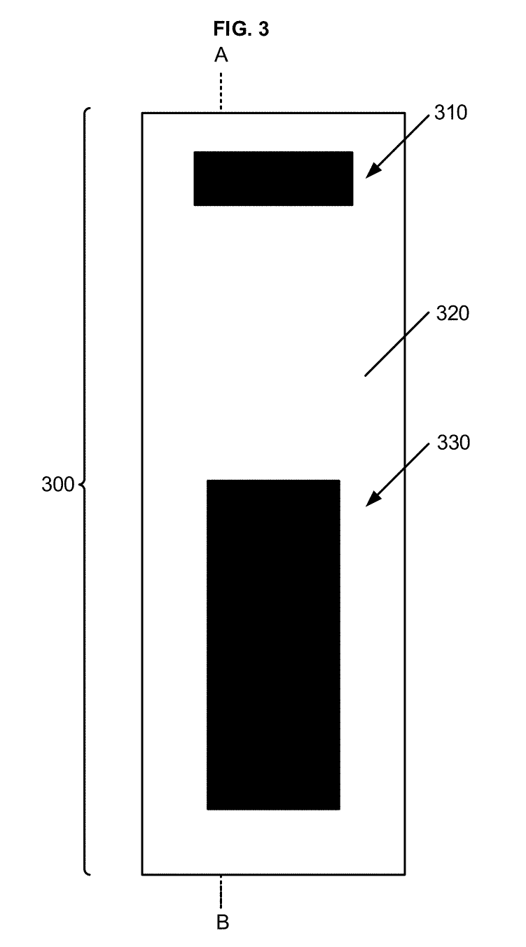

FIG. 3 depicts a representative external view of an immunoassay device, such as a lateral flow assay device 300. The immunoassay device, such as a lateral flow assay device, is intended to be single-use, low cost and disposable. The lateral flow assay device 300 is illustrated as a top down view. The lateral flow assay device 300 includes an optional cover 320, such as a plastic cover of a size, shape and position to maintain the relative position of the internal components and protect them from contamination. During use, the cover 320 also serves to prevent leakage of a potentially infectious human serology sample, such as blood or urine, from the interior of the lateral flow assay device 300. The lateral flow assay device 300 includes a sample-addition aperture 310 adjacent to a first end of the device. The sample-addition aperture 310 is of a size, shape and position to allow sample fluid to be added to the interior of the device, for example drops of blood. In some embodiments the sample-addition aperture 310 is of a size, shape and position to allow assay buffer or wash fluid to be added to the interior of the device.

A lateral flow assay device 300 also includes a visualization region 330. The visualization region 330 corresponds with a detection region interior to the device. In some embodiments, the visualization region 330 includes an aperture in the cover 320 corresponding with an appropriate position on the adjacent internal membrane. A clear plastic cover can be added to create a window in the device for the visualization region 330.

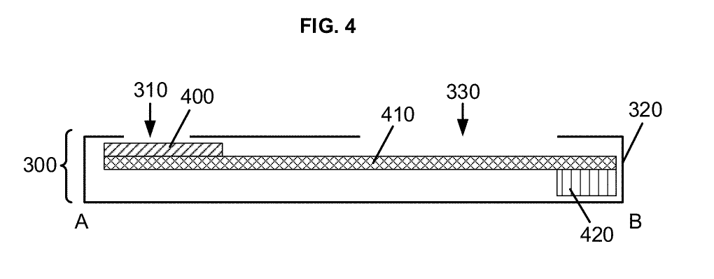

FIG. 4 depicts a cross-section view of a lateral flow assay device 300 such as shown in FIG. 3. The view of FIG. 4 corresponds with the long axis of the device, from point A to point B in the figures. The lateral flow assay device 300 includes a cover 320 substantially surrounding the exterior of the device 300. The lateral flow assay device 300 includes a sample-addition aperture 310 adjacent to a first end of the device 300. A sample pad 400 is positioned adjacent to the sample-addition aperture 310. The sample pad 400 is fabricated from a material absorbent to the sample. In some embodiments, the sample pad 400 includes a structure that filters part of a sample, for example larger particles from blood or urine. The sample pad 400 is positioned and configured to receive a sample at a first side adjacent to the sample-addition aperture 310 and to permit the sample to exit the sample pad 400 at a second side positioned adjacent to a conjugate pad 410. Some embodiments include a sample pad that includes multiple sub-parts, such as multiple layers or regions. Some embodiments include an additional component, such as a filter or selective membrane, positioned in alignment with the sample pad. For example a selectively permeable membrane can be positioned adjacent to the sample pad between the sample pad and the conjugate pad 410.

A conjugate pad 410 is positioned along the length of the lateral flow assay device 300, with a first end positioned adjacent to the sample pad 310 and a second end positioned adjacent to a waste pad 420. In some embodiments, a detection complex able to bind anti-ETRAMP5 antibodies is positioned within the sample pad. In some embodiments, a detection complex able to bind anti-ETRAMP5 antibodies is positioned within the conjugate pad at a position adjacent to the sample pad. Additional features, such as salts, assay positive control components, assay negative control components, and/or flow control elements can be included in the conjugate pad. Although the conjugate pad illustrated in FIG. 4 is shown as a single unit, in some embodiments a conjugate pad can be made up of multiple sub parts, such as layers or regions fabricated from different materials.

A waste pad 420 is positioned adjacent to the second end of the conjugate pad. In the illustrated embodiment, the waste pad 420 is positioned adjacent to the lower surface of the second end of the conjugate pad, in a position to permit excess fluid to flow downward into the waste pad. The waste pad is of a size, shape and material to retain excess fluid within the device to minimize the possibility of cross-contamination or infection spread from fluid leakage. Although the waste pad is illustrated as a single unit herein, in some embodiments it is formed from multiple layers or regions of the same or different materials.

In some embodiments, one or more areas of the second end of the conjugate pad include one or more areas impregnated with a construct protein including fragments from a malaria parasite protein expressed during early infection of a human host. For example the construct protein can include one or more fragments of AMA1, GLURP, MSP1.19, Rh2030, EBA181, EBE175, Etramp4Ag2 MSP2, HSP40, GexP, Hyp2, SBP1, SEA, H101 and hSG6 proteins expressed as a construct protein.

In some embodiments, an immunoassay device for detection of recent malaria infection includes: a sample pad positioned adjacent to a first end of a conjugate pad, wherein either the sample pad or the first end of the conjugate pad include a first detection complex able to bind human anti-ETRAMP5 antibodies and a second detection complex able to bind to a malaria immune response antigen; wherein a second end of the conjugate pad includes a first region including a first capture particle complexed with a complex molecule including ETRAMP5 protein fragments, and a second region including a second capture particle complexed with a complex molecule including one or more fragments of the malaria immune response protein. The first and second regions of the conjugate pad can be positioned as distinct regions of the pad, for example as distinct lines or bands across the width of the pad. Depending on the embodiment, a positive result for the assay can include detection of a reaction (e.g. a color change) in both the first and second regions of the conjugate pad. Some embodiments also include a positive control test line, wherein a positive result is indicated by both the first and second regions of the conjugate pad as well as the positive control test line all showing a positive result, for example a color change.

A first or second detection complex can include a capture particle complexed with anti-IgG antibodies. In some embodiments, either a first or a second detection complex can include a capture particle complexed with anti-IgG antibodies. In some embodiments, only one of a first or a second detection complex will include a capture particle complexed with anti-IgG antibodies. Some embodiments include a capture particle complexed with a complex molecule including ETRAMP5 protein fragments. For example, the complex molecule including ETRAMP5 protein fragments can include multiple ETRAMP5 protein fragments, attached to each other in series. The ETRAMP5 protein fragments can correspond to the same section of the full molecule, for example multiple copies of expressed exon 1 of ETRAMP5 attached in series. The ETRAMP5 protein fragments can correspond to two or more different sections of the full molecule, for example attached in series.

In some embodiments, the second detection complex able to bind to a malaria immune response antigen includes at least one of: carbohydrate-binding module family 2 (CBM2), gametocyte exported protein (GEXP), merozoite surface protein 1 (MSP1), merozoite surface protein 2 (MSP2), glutamate-rich protein (GLURP), circumsporozoite protein (CSP), and/or reticulocyte-binding protein (Rh5). The protein may be a fragment or portion of the full malaria immune response antigen protein, for example MSP2 can include one or more of the MSP2.CH150.0 and/or MSP2.Dd2.KT fragments of MSP2. There is a second capture particle complexed with a complex molecule including one or more fragments of the malaria immune response protein, which will correspond with the immune response antigen from the second detection complex. For example wherein the malaria immune response antigen includes the carbohydrate-binding module family 2 (CBM2) protein or a fragment thereof, the second capture particle complexed with a complex molecule includes the carbohydrate-binding module family 2 (CBM2) protein.

Some embodiments include a third detection complex, and a third portion of the conjugate pad including a capture particle corresponding to the third detection complex. For example the third detection complex can include a malaria immune response antigen from the list provided above, and a third portion of the conjugate pad including a capture particle complexed with a complex molecule including one or more fragments of the malaria immune response protein.

In some embodiments, an immunoassay device for recent malaria infection includes: a sample pad positioned adjacent to a first end of a conjugate pad, wherein either the sample pad or the first end of the conjugate pad include a first detection complex able to bind human anti-ETRAMP5 antibodies and a second detection complex able to bind to a malaria immune response antigen; and an analytical membrane in contact with a second end of the conjugate pad, the analytical membrane including a first region including a first capture particle complexed with a complex molecule including ETRAMP5 protein fragments, and a second region including a second capture particle complexed with a complex molecule including one or more fragments of the malaria immune response protein.

The analytical membrane can be of a type used for immunoassays, in particular lateral flow assays. The analytical membrane used in an embodiment can be selected based on factors such as cost, durability, stability, flow rate through the membrane and ability to attach to the detection complexes.

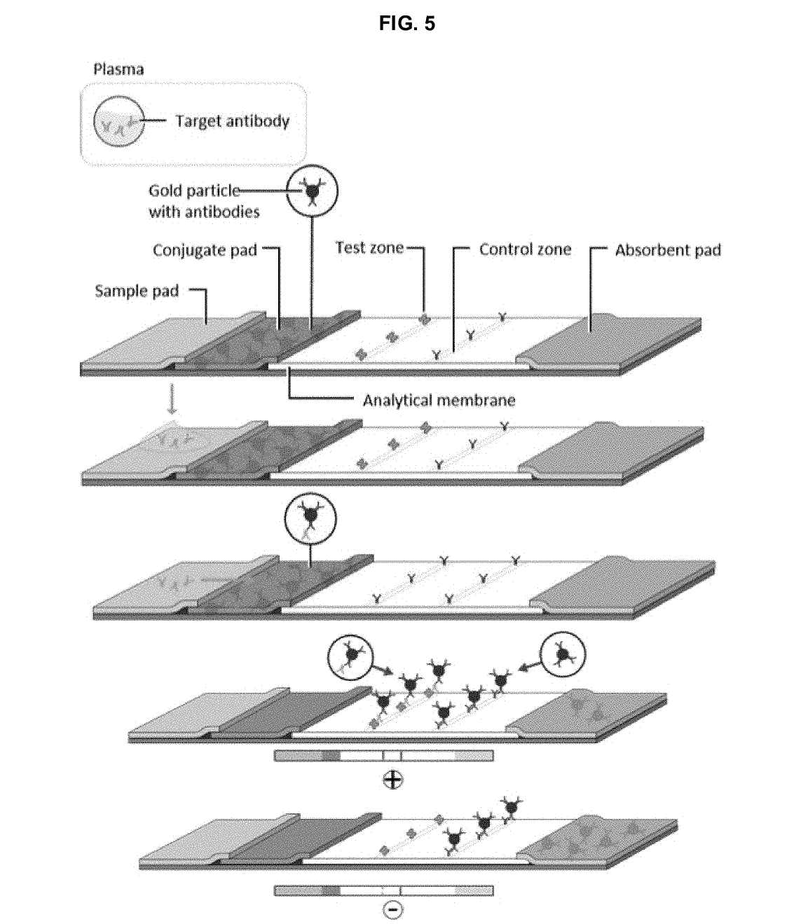

FIG. 5 depicts a schematic of an immunoassay in a lateral flow assay format. The exterior housing of the lateral flow assay has been removed from the schematic for better visualization of the interior. The topmost diagram shows a lateral flow assay prior to use, with a sample pad at the left side and on top of the end of a conjugate pad. The conjugate pad is impregnated with at least one detection complex. In the illustrated example the detection particle is formed of a gold particle complexed with antibodies against a malaria immune response antigen, for example ETRAMP5. The conjugate pad is positioned in direct contact with the sample pad, with the first end of the conjugate pad (to the left side in the view of FIG. 5) positioned underneath the edge of the sample pad. An analytical membrane is positioned adjacent to and in contact with the second end of the conjugate pad. The analytical membrane includes at least one test zone as well as a control zone. The test zone has affixed particles of a first capture particle complexed with a complex molecule including malaria antigen proteins. For example, at least one test zone includes ETRAMP5 protein fragments affixed to the analytical membrane. The individual first capture particles can be positioned in a line across the width of the analytical membrane (as shown in FIG. 5) or as a dot, circle, line otherwise positioned relative to the analytical membrane, or other pattern. The analytical membrane includes a control zone including antibodies to a control molecule affixed to the analytical membrane. The individual antibodies to a control molecule can be positioned in a line across the width of the analytical membrane (as shown in FIG. 5) or as a dot, circle, line otherwise positioned relative to the analytical membrane, or other pattern. In some embodiments the control zone and at least one test zone intersect, for example forming an “X” or similar symbol with crossed lines. An absorbent pad, sometimes referred to as a waste pad, is positioned distal to the far end of the analytical membrane from the conjugate pad.

The second schematic from the top in FIG. 5 illustrates plasma, for example from a blood sample, being deposited on the top surface of the sample pad. Some embodiments include additional filter layers positioned on top of the sample pad, the filter layers removing red blood cells and cellular debris particles from the sample prior to the fluid sample entering the sample pad. If the plasma sample comes from a person recently infected with malaria, the sample will contain antibodies to malaria antigens corresponding to the infecting parasite. FIG. 5 illustrates these antibodies as Y-shaped molecules within the plasma.

The center schematic of FIG. 5 illustrates that after the plasma sample integrates into the sample pad, antibodies within the plasma, including possible antibodies to malaria antigens, move with fluid flow from the sample pad into the conjugate pad. In the conjugate pad, any antibodies present in the sample can bind with any detection complexes in the conjugate pad. This antibody-detection complex interaction and potential binding depends on the present of correlating antibodies and detection complexes. For example an antibody to the malaria protein ETRAMP5 could interact with and bind to a detection complex containing an ETRAMP5 protein fragment including an epitope for that antibody. If no antibody corresponding to the detection particle is present in the plasma sample, no specific binding to the detection complexes would occur in that instance. The illustration shows a situation where the patient who provided the blood sample has antibodies to the detection particle.

The two lower portions of FIG. 5 illustrate the basis for a positive (indicated as +) and a negative (indicated as −) result. The positive result is the upper illustration, second from the bottom in FIG. 5, and the negative result is the lowest portion of the illustration. In the situation where a person providing a blood sample has antibodies in their blood which recognize and bind to proteins on the detection complex, for example corresponding antibodies. The larger complex will then move, through fluid flow, from the conjugate pad into the analytical membrane. If the antibodies in the larger detection complex recognize and bind to a protein affixed in a test zone, the detection complexes will cluster in that area and form a visible color change in the analytical membrane. A control detection particle also present in the conjugate pad is also moved, through fluid flow, into the analytical membrane and binds to molecules affixed in the control zone of the analytical membrane. The control detection particles form a visible color change at the control zone. In the positive assay, both the test zone and the control zone have detection particle binding and two lines form on the analytical membrane. In the negative assay, only the control detection complex binds and a single line is formed. For both results, excess detection complexes move with fluid flow into the absorbent pad at the far right of the illustrated assays.

In some embodiments, an immunoassay includes two or more test zones and corresponding types of detection particles with molecules that will bind with multiple blood indicators of recent infection. For example an immunoassay might include a test zones and corresponding type of detection particles recognizing ETRAMP5 as well as a non-ETRAMP5 malaria immune response antigen. For example a non-ETRAMP5 malaria immune response antigen could include one of: carbohydrate-binding module family 2 (CBM2), gametocyte exported protein (GEXP), merozoite surface protein 1 (MSP1), merozoite surface protein 2 (MSP2), glutamate-rich protein (GLURP), circumsporozoite protein (CSP), and/or reticulocyte-binding protein (Rh5). For immunoassays including two or more test zones, a positive result would be scored as all of the test lines changing color (i.e. detection of all of the malaria infection indicator targets) as well as the control line changing color. Any less than all of the zones changing color would be scored as a negative result. The addition of a second, or in some examples a third, test line would increase the specificity of the assay. This is clinically useful for a complex clinical situation, such as is present in a population living in a malaria-endemic region.

### EXAMPLES

### Example 1. Molecular and Biochemical Engineering of Etramp 5 ag1 Variants

Earlier testing showed a level of non-specific binding, potentially leading to false positive results, that it was desirable to minimize. A multimerised, tag-less version of the Etramp 5 ag1 protein would eliminate potential non-specific binding and potentially increase the serological signal due to the increase in the presentation of epitopes resulting from the multimerisation of the antigen. The aim was to both reduce or eliminate non-specific binding as well as improve the serological signal by immunoassay, including lateral flow assay.

Six constructs were designed and expressed. The constructs included three variants using the pET/His tag expression system and three variants using the pGEX/GST tag expression system. Each construct engineered to contain, in addition to the endogenous plasmid derived purification tag:
1. A BirA biotin ligation site has been engineered to lie adjacent to the Etramp 5 ag 1 sequence to allow the expressed protein to be ligated via a biotin ligation reaction and allowing the multimerisation of the antigen to take place.
2. A cTPR[X] linker sequence (consensus tetratricopeptide repeat sequence with X referring to the number of repeats). Linkers sequences containing three (cTPR3) or twelve (cTPR12) repeats were used. The aim being to provide distance between the purification tag (specifically for the GST variants) and the BirA tag allowing the biotin ligation reaction to proceed unimpeded by stearic hindrance. The concern is that the GST, being the larger component of the construct could potentially block access of the ligase to the tag.
3. Finally, a HRV 3C protease site was also engineered into the constructs to lie between the linker and the BirA site. This will allow the linker sequence and the purification tag to be cleaved off if required, following successful biotin mediated ligation of the Etramp 5 ag1 protein.

The linker is intended to provide ‘space’ between the GST and the BirA tag allowing access of the ligase to the tag.

Expression Constructs:

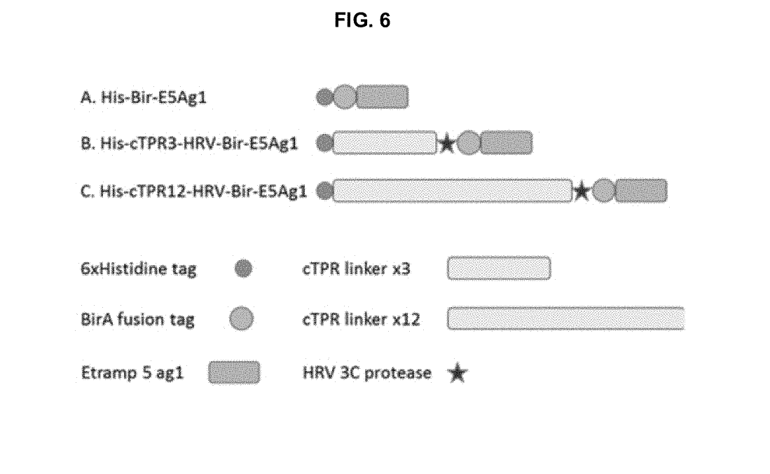

FIG. 6 illustrates a schematic summary of the Etramp 5 ag1 His-tagged variants highlighting the positions of the engineered components in relation to the Etramp 5 ag 1 sequence. Regions in grey highlight the cTPR3 (short) or cTPR12 (long) linker sequence. Italicized sequence denotes HRV 3C protease site (not required for the GST-Bir-E5Ag1 construct). Region in bold highlights the BirA biotin ligation site. The underlined region highlights the Etramp 5 ag1 sequence.

Fig 6 row A
His-Bir-E5Ag1
Restriction site- CATATG
GLNDIFEAQKIEWHEQLDMGSVHNNNSVVGNSSSHSPSSSSSSPSSSSSS
SSSSPSASSSSSSSSPASSSSSPSSTSDDSKNASLDKIDEELQKKKKNEK
L
Stop codon-TAA
Restriction site-GGATCC
Fig 6 row B.
His-cTPR3-HRV-Bir-E5Ag1
Restriction site- CATATG

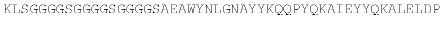

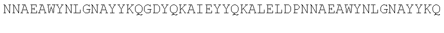

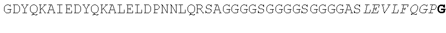

LNDIFEAQKIEWHEQLDMGSVHNNNSVVGNSSSHSPSSSSSSPSSSSSSS
SSSPSASSSSSSSSPASSSSSPSSTSDDSKNASLDKIDEELQKKKKNEKL
Stop codon-TAA
Restriction site-GGATCC
Fig 6 row C.
His-cTPR12-HRV-Bir-E5Ag1
Restriction site- CATATG

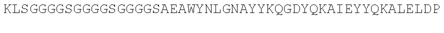

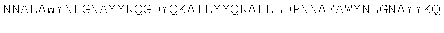

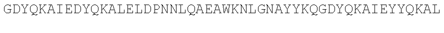

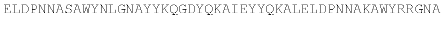

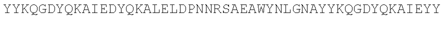

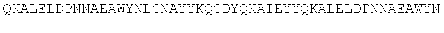

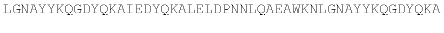

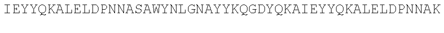

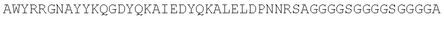

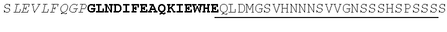

SSPSSSSSSSSSSPSASSSSSSSSPASSSSSPSSTSDDSKNASLDKIDEE
LQKKKKNEKL
Stop codon-TAA
Restriction site-GGATCC

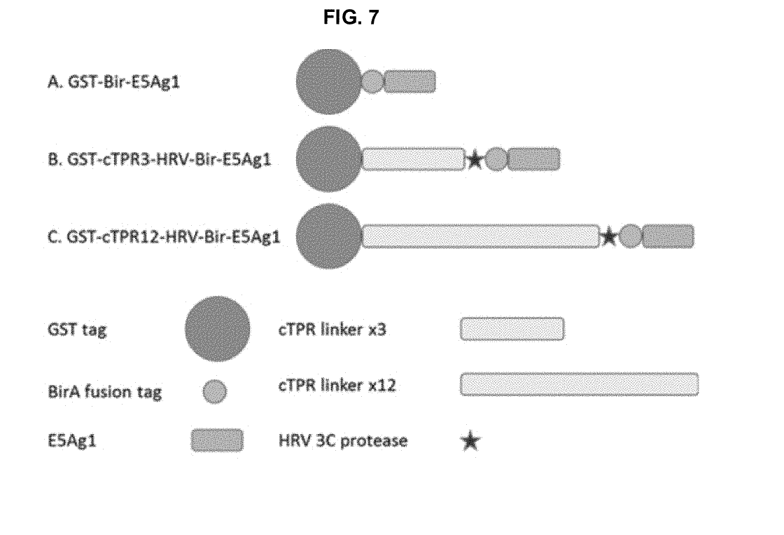

FIG. 7 illustrates a summary of the Etramp 5 ag1 GST-tagged variants highlighting the positions of the engineered components in relation to the Etramp 5 ag 1 sequence. Regions in grey highlight the cTPR12 linker sequence. Italicized sequence denotes HRV 3C protease site (not required for the GST-Bir-E5Ag1 construct). Region in bold highlights the BirA biotin ligation site. The underlined region highlights the Etramp 5 ag1 sequence.

Fig 7 row A.
GST-Bir-E5Ag1
Restriction site- GAATTC
GLNDIFEAQKIEWHEQLDMGSVHNNNSVVGNSSSHSPSSSSSSPSSSSSS
SSSSPSASSSSSSSSPASSSSSPSSTSDDSKNASLDKIDEELQKKKKNEK
L
Stop codon-TAA
Restriction site- GTCGAC
Fig 7 row B.
GST-cTPR3-HRV-Bir-E5Ag1
Restriction site- GAATTC

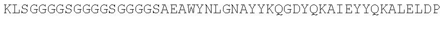

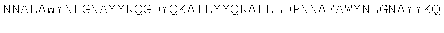

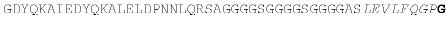

LNDIFEAQKIEWHEQLDMGSVHNNNSVVGNSSSHSPSSSSSSPSSSSSSS
SSSPSASSSSSSSSPASSSSSPSSTSDDSKNASLDKIDEELQKKKKNEKL
Stop codon-TAA
Restriction site- GTCGAC
Fig 7 row C.
GST-cTPR12-HRV-Bir-E5Ag1
Restriction site- GAATTC

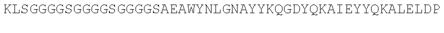

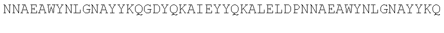

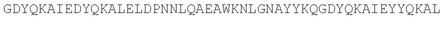

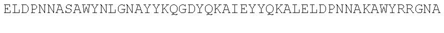

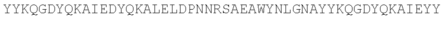

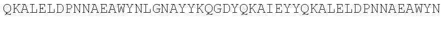

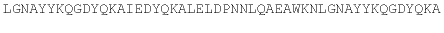

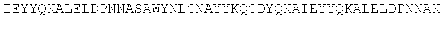

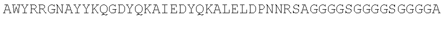

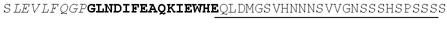

SSPSSSSSSSSSSPSASSSSSSSSPASSSSSPSSTSDDSKNASLDKIDEE
LQKKKKNEKL
Stop codon-TAA
Restriction site- GTCGAC

### Example 2. Malaria Antigen Test Results in Populations

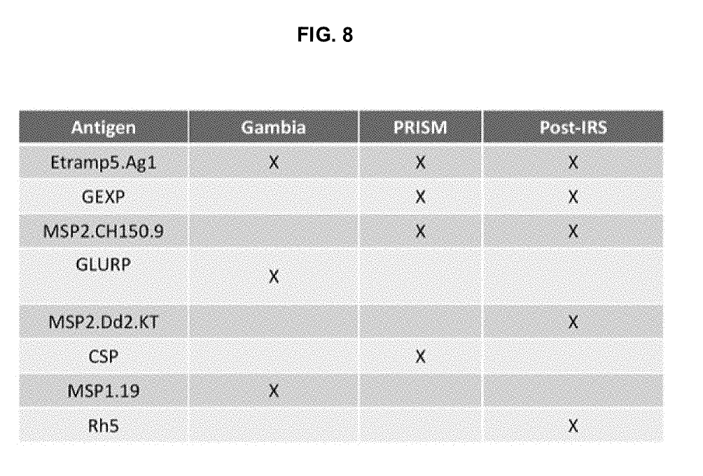

FIG. 8 illustrates a summary of serum malaria assay results taken from multiple individuals under 15 years of age at three test sites in Africa with endemic malaria. Each malaria antigen was tested in each serum sample with the Luminex assay (see Helb et al., Novel Serologic Biomarkers Provide Accurate Estimates of Recent Plasmodium Falciparum Exposure for Individuals and Communities, PNAS doi 10.1073, published online Jul. 27, 2015, which is incorporated by reference). Each sample was also tested by microscopy for the presence of malaria parasites. A positive for each antigen was scored when a sample was positive for malaria parasites by microscopy and also tested positive for that antigen. An X indicates that the particular antigen scored high relative to other antigens in individuals testing positive for malaria.

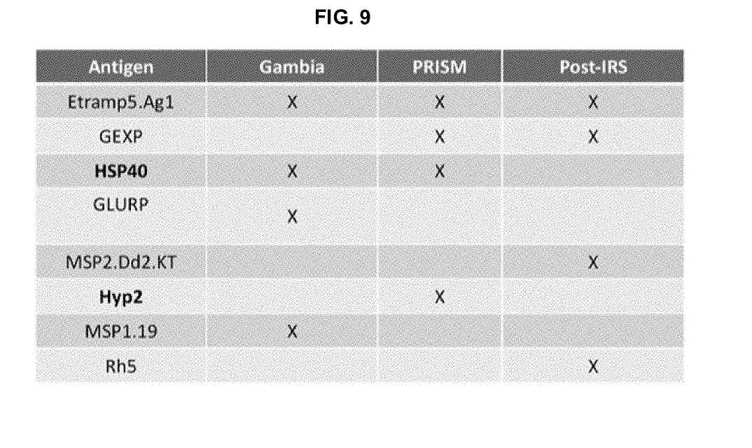

FIG. 9 illustrates a summary of serum malaria assay results taken from multiple individuals of all ages tested at the test sites in Africa with endemic malaria. Testing protocols were as described above and in Helb, ibid., which is incorporated by reference.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. An immunoassay device for recent malaria infection, comprising:
a sample pad positioned adjacent to a first end of a conjugate pad, wherein either the sample pad or the first end of the conjugate pad include a detection complex able to bind anti-ETRAMP5 antibodies;
wherein a second end of the conjugate pad includes one or more areas impregnated with a construct protein including ETRAMP5 protein fragments.

2. The immunoassay device of claim 1, wherein the detection complex for anti-ETRAMP5 antibodies comprises:
a capture particle complexed with anti-IgG antibodies.

3. The immunoassay device of claim 1, wherein the detection complex for anti-ETRAMP5 antibodies comprises:
a capture particle complexed with a complex molecule including ETRAMP5 protein fragments.

4. The immunoassay device of claim 3, wherein the detection complex for anti-ETRAMP5 antibodies comprises:
a capture particle complexed with a complex molecule including multiple ETRAMP5 protein fragments attached to each other in a series.

5. The immunoassay device of claim 1, wherein the detection complex for anti-ETRAMP5 antibodies comprises:
a detection complex for human anti-ETRAMP5 antibodies.

6. The immunoassay device of claim 1, wherein the detection complex for anti-ETRAMP5 antibodies comprises:
a first capture particle complexed with a complex molecule including ETRAMP5 protein fragments; and
a second capture particle complexed with a complex molecule including a non-ETRAMP5 malaria immune response antigen.

7. The immunoassay device of claim 6, wherein the non-ETRAMP5 malaria immune response antigen comprises at least one of: carbohydrate-binding module family 2 (CBM2), gametocyte exported protein (GEXP), merozoite surface protein 1 (MSP1), merozoite surface protein 2 (MSP2), glutamate-rich protein (GLURP), circumsporozoite protein (CSP), and/or reticulocyte-binding protein (Rh5).

8. The immunoassay device of claim 6, wherein the complex molecule including ETRAMP5 protein fragments comprises a series of ETRAMP5 protein fragments.

9. The immunoassay device of claim 1, wherein the construct protein including ETRAMP5 protein fragments comprises:
a protein corresponding to exon 1 of the ETRAMP5 DNA sequence.

10. The immunoassay device of claim 1, wherein the construct protein including ETRAMP5 protein fragments comprises:
a protein corresponding to exon 1 of the ETRAMP5 genomic DNA sequence and a Glutathione S-transferase (GST) protein from Schistosoma japonicum.

11. The immunoassay device of claim 1, wherein the construct protein including ETRAMP5 protein fragments comprises:
a protein corresponding to exon 1 of the ETRAMP5 genomic DNA sequence and a human Glutathione S-transferase (GST) protein.

12. The immunoassay device of claim 1, wherein the construct protein including ETRAMP5 protein fragments comprises:
a protein corresponding to exon 1 of the ETRAMP5 genomic DNA sequence and at least six histidine molecules.

13. The immunoassay device of claim 1, wherein the construct protein including ETRAMP5 protein fragments comprises:
a protein corresponding to exon 1 of the ETRAMP5 genomic DNA sequence and a tag protein used as a tag molecule.

14. The immunoassay device of claim 1, wherein the construct protein including ETRAMP5 protein fragments comprises:
a protein corresponding to exon 1 of the ETRAMP5 genomic DNA sequence and a protein including a portion of carbohydrate binding molecule 2 (CBM2) protein.

15. The immunoassay device of claim 1, wherein the construct protein including ETRAMP5 protein fragments is purified from E. Coli.

16. The immunoassay device of claim 1, wherein the construct protein including ETRAMP5 protein fragments is purified from mammalian cells.

17. An immunoassay device for recent malaria infection, comprising:
a sample pad positioned adjacent to a first end of a conjugate pad, wherein either the sample pad or the first end of the conjugate pad include a first detection complex able to bind human anti-ETRAMP5 antibodies and a second detection complex able to bind to a malaria immune response antigen;
wherein a second end of the conjugate pad includes a first region including a first capture particle complexed with a complex molecule including ETRAMP5 protein fragments, and a second region including a second capture particle complexed with a complex molecule including one or more fragments of the malaria immune response protein.

18. The immunoassay device of claim 17, wherein the first or the second detection complex comprises:
a capture particle complexed with anti-IgG antibodies.

19. The immunoassay device of claim 17, wherein the first detection complex comprises:
a capture particle complexed with a complex molecule including ETRAMP5 protein fragments.

20. The immunoassay device of claim 17, wherein the second detection complex comprises at least one of: carbohydrate-binding module family 2 (CBM2), gametocyte exported protein (GEXP), merozoite surface protein 1 (MSP1), merozoite surface protein 2 (MSP2), glutamate-rich protein (GLURP), circumsporozoite protein (CSP), and/or reticulocyte-binding protein (Rh5).

21. The immunoassay device of claim 17, wherein the first capture particle complexed with a complex molecule including ETRAMP5 protein fragments comprises:
a protein corresponding to exon 1 of the ETRAMP5 DNA sequence.

22. The immunoassay device of claim 17, wherein the first capture particle complexed with a complex molecule including ETRAMP5 protein fragments comprises:
a protein corresponding to exon 1 of the ETRAMP5 genomic DNA sequence and a Glutathione S-transferase (GST) protein from Schistosoma japonicum.

23. The immunoassay device of claim 17, wherein the first capture particle complexed with a complex molecule including ETRAMP5 protein fragments comprises:
a protein corresponding to exon 1 of the ETRAMP5 genomic DNA sequence and a human Glutathione S-transferase (GST) protein.

24. The immunoassay device of claim 17, wherein the first capture particle complexed with a complex molecule including ETRAMP5 protein fragments comprises:
a protein corresponding to exon 1 of the ETRAMP5 genomic DNA sequence and at least six histidine molecules.

25. The immunoassay device of claim 17, wherein the first capture particle complexed with a complex molecule including ETRAMP5 protein fragments comprises:
a protein corresponding to exon 1 of the ETRAMP5 genomic DNA sequence and a tag protein used as a tag molecule.

26. The immunoassay device of claim 17, wherein the first capture particle complexed with a complex molecule including ETRAMP5 protein fragments includes a protein corresponding to exon 1 of the ETRAMP5 genomic DNA sequence, the second capture particle complexed with a complex molecule including one or more fragments of the malaria immune response protein includes a fragment of carbohydrate binding molecule 2 (CBM2) protein.

27. The immunoassay device of claim 17, wherein the second capture particle complexed with a complex molecule including one or more fragments of the malaria immune response protein comprises one or more of: carbohydrate-binding module family 2 (CBM2), gametocyte exported protein (GEXP), merozoite surface protein 1 (MSP1), merozoite surface protein 2 (MSP2), glutamate-rich protein (GLURP), circumsporozoite protein (CSP), and/or reticulocyte-binding protein (Rh5) protein fragments.

28. The immunoassay device of claim 17, wherein at least one of the ETRAMP5 protein fragments and/or the one or more fragments of the malaria immune response protein are purified from E. Coli.

29. The immunoassay device of claim 17, wherein at least one of the ETRAMP5 protein fragments and/or the one or more fragments of the malaria immune response protein are purified from mammalian cells.

30. An immunoassay device for recent malaria infection, comprising:
a sample pad positioned adjacent to a first end of a conjugate pad, wherein either the sample pad or the first end of the conjugate pad include a first detection complex able to bind human anti-ETRAMP5 antibodies and a second detection complex able to bind to a malaria immune response antigen; and
an analytical membrane in contact with a second end of the conjugate pad, the analytical membrane including a first region including a first capture particle complexed with a complex molecule including ETRAMP5 protein fragments, and a second region including a second capture particle complexed with a complex molecule including one or more fragments of the malaria immune response protein.

31. The immunoassay device of claim 30, wherein the first or the second detection complex comprises:
a capture particle complexed with anti-IgG antibodies.

32. The immunoassay device of claim 30, wherein the first detection complex comprises:
a capture particle complexed with a complex molecule including ETRAMP5 protein fragments.

33. The immunoassay device of claim 30, wherein the second detection complex comprises at least one of: carbohydrate-binding module family 2 (CBM2), gametocyte exported protein (GEXP), merozoite surface protein 1 (MSP1), merozoite surface protein 2 (MSP2), glutamate-rich protein (GLURP), circumsporozoite protein (CSP), and/or reticulocyte-binding protein (Rh5).

34. The immunoassay device of claim 30, wherein the first capture particle complexed with a complex molecule including ETRAMP5 protein fragments comprises:
a protein corresponding to exon 1 of the ETRAMP5 DNA sequence.

35. The immunoassay device of claim 30, wherein the first capture particle complexed with a complex molecule including ETRAMP5 protein fragments comprises:
a protein corresponding to exon 1 of the ETRAMP5 genomic DNA sequence and a Glutathione S-transferase (GST) protein from Schistosoma japonicum.

36. The immunoassay device of claim 30, wherein the first capture particle complexed with a complex molecule including ETRAMP5 protein fragments comprises:
a protein corresponding to exon 1 of the ETRAMP5 genomic DNA sequence and a human Glutathione S-transferase (GST) protein.

37. The immunoassay device of claim 30, wherein the first capture particle complexed with a complex molecule including ETRAMP5 protein fragments comprises:
a protein corresponding to exon 1 of the ETRAMP5 genomic DNA sequence and at least six histidine molecules.

38. The immunoassay device of claim 30, wherein the first capture particle complexed with a complex molecule including ETRAMP5 protein fragments comprises:
a protein corresponding to exon 1 of the ETRAMP5 genomic DNA sequence and a tag protein used as a tag molecule.

39. The immunoassay device of claim 30, wherein the first capture particle complexed with a complex molecule including ETRAMP5 protein fragments includes a protein corresponding to exon 1 of the ETRAMP5 genomic DNA sequence, the second capture particle complexed with a complex molecule including one or more fragments of the malaria immune response protein includes a fragment of carbohydrate binding molecule 2 (CBM2) protein.

40. The immunoassay device of claim 30, wherein the second capture particle complexed with a complex molecule including one or more fragments of the malaria immune response protein comprises one or more of: carbohydrate-binding module family 2 (CBM2), gametocyte exported protein (GEXP), merozoite surface protein 1 (MSP1), merozoite surface protein 2 (MSP2), glutamate-rich protein (GLURP), circumsporozoite protein (CSP), and/or reticulocyte-binding protein (Rh5) protein fragments.

41. The immunoassay device of claim 30, wherein at least one of the ETRAMP5 protein fragments and/or the one or more fragments of the malaria immune response protein are purified from E. Coli.

42. The immunoassay device of claim 30, wherein at least one of the ETRAMP5 protein fragments and/or the one or more fragments of the malaria immune response protein are purified from mammalian cells.
